(19) United States
(12) Patent Application Publication
(10) Pub. No.: US 2014/0234804 A1
(43) Pub. Date: Aug. 21, 2014

(54) Assisted Guidance and Navigation Method in Intraoral Surgery

(71) Applicants: Ta-Ko Huang, Kaohsiung City (TW); Zong-Han Lyu, Kaohsiung City (TW); Yu-Hsin Hsieh, Kaohsiung City (TW)

(72) Inventors: Ta-Ko Huang, Kaohsiung City (TW); Zong-Han Lyu, Kaohsiung City (TW); Yu-Hsin Hsieh, Kaohsiung City (TW)

(73) Assignee: EPED INC., Kaohsiung City (TW)

(21) Appl. No.: 14/347,225

(22) PCT Filed: May 2, 2012
(86) PCT No.: PCT/CN2012/074990

§ 371 Date: Mar. 25, 2014

(51) Int. Cl. A61B 6/14; A61C 1/08; A61B 6/00; A61B 6/03

(52) U.S. Cl. CPC A61B 6/14; A61B 6/032; A61C 1/082; A61B 6/54; A61B 6/52
USPC 433/215

(57) ABSTRACT

An assisted guidance and navigation method in intraoral surgeries is a method using computerized tomography (CT) photography and an optical positioning system to track medical appliances, the method including: first providing an optical positioning treatment instrument and an optical positioning device; then obtaining image data of the intraoral tissue receiving treatment through CT photography, precisely displaying actions of the treatment instrument in the image data, and real-time checking an image and guidance and navigation. Therefore, during the surgery, the existing use habits of the physicians are not affected and accurate and convenient auxiliary information is provided, and attention is paid to using the treatment instrument in physical environments such as a patient's tooth or dental model.

### BACKGROUND OF THE INVENTION

1. Field of the Invention

The present invention relates to assisted guidance and navigation in intraoral surgeries, especially an assisted guidance and navigation method for performing intraoral surgeries accurately and quickly through assistance of an optical positioning device.

2. Related Art

Preoperative assessment and planning is very important in the existing intraoral surgical treatment techniques, and in order to have a better understanding of the status of the patient's intraoral tissue, a doctor often needs to refer to different information. For example, the tooth model may provide appearance information of the patient's teeth, which may allow the doctor to understand the patient's dental occlusion condition; computerized tomography (CT) photography provides anatomical information inside the mouth, including the patient's teeth and jaw bone status, the information is very important for some intraoral surgeries, such as tooth implant surgery, root canalling, impacted tooth extraction and temporomandibular joint assessment.

By taking the tooth implant surgery as an example, in order to make the edentulous patient's teeth to recover the normal occlusion function, artificial tooth implant is one of the treatment manners, the direction and location of the tooth implant plays an important role in the results for treatment, improper orientation may cause excessive occlusal stress, resulting in rapid loss of bones of bony ridges, and falling off of the implant.

Proper orientation of tooth implant depends on two factors, i.e., perfect preoperative planning and precise drilling during surgery. In the preoperative planning of the existing tooth implant, the doctor must use the patient's dental model and CT photography as the basis of planning optimal implant orientation, the dental model mainly provides external information, so that the doctor could understand the patient's upper and lower teeth occlusion and provide postoperative appearance information; conversely, CT photography may provide internal anatomical information, including teeth, jaw bone, alveolar bone nerves, upper nasal sinus, and so on. The implantation of implant during surgery, currently, mostly relies on the clinical experience and surgical techniques of the doctor, to implement the preoperative planning into the patient's mouth, so the doctor's freehand drilling stability and visual three-dimensional space mastery becomes an important factor of the quality of surgery.

Also, integration of the CT photography and the surgical guide plate also begins to be used in the teeth implant surgery, to enhance the quality of treatment. The CT photography provides a physical 1:1 patient oral tissue image without distortion, which enables the doctor to plan the surgery more accurately through cooperating with 3D imaging software, then the treatment plan is sent to the surgical template manufacturing system, the resultant surgical guide plate can precisely guide the surgical drill, allowing the doctor to place the implant at the best position. The surgical guide plate is usually made of resin materials to sit across braces adjacent to the teeth, a metal guide outer ring is mounted at an implant area hole, sleeves with different apertures are inserted to guide drills with different sizes during the surgery, and the sizes gradually expand to the size of the aperture of the implant into the jaw bone, which not only provides stability required by the doctor, but also implements the optimal implant orientation of the preoperative planning.

Although the integration of the CT photography and the surgical guide plate provides the manner of treating intraoral tissues, it is not widely used clinically. One major reason is that using the 3D imaging software to plan a surgery is not a way the doctors are familiar to. In fact, the doctors spend most of the time in using instruments in physical environments such as a patient's tooth or dental model, and use general computer input devices (such as mouse and keyboard) to plan the dental treatment manner for the preoperative planning, for example, direction, angle and depth of the implant of the preoperative planning, and selection of treatment instruments, but the mouse is used to operate complex 3D virtual software during the treatment, to make the system provide guide and alert according to planning data, and the doctors often feel quite helpless and hesitant.

### SUMMARY OF THE INVENTION

To solve the above defects, an objective of the present invention is to provide an assisted guidance and navigation method in intraoral surgeries, which uses CT photography and an optical positioning device to decide relevant direction, angle and depth for a surgery, also provides selection information of treatment instruments, and real-time provides CT images of intraoral tissues around the treatment instruments, to achieve the function of assisting guidance and navigation during the surgery.

Another objective of the present invention is to provide an assisted guidance and navigation method in intraoral surgeries, which uses CT photography and an optical positioning device to make the doctor, during intraoral surgical treatment, real-time check image data and guide a surgery of treating the intraoral tissue, and pay attention to using the treatment instrument in physical environments such as a patient's tooth or dental model.

To achieve the above objectives, the technical solution of the present invention is implemented as follows:

an assisted guidance and navigation method in intraoral surgeries, which is a method using CT photography and an optical positioning system to track medical appliances, the method at least including: step (A) providing an optical positioning treatment instrument and an optical positioning device at an intraoral tissue receiving treatment; step (B) obtaining image data of the intraoral tissue receiving treatment through CT photography, obtaining a positioning relationship between the treatment instrument and the optical positioning device, making the image data and physical space generate correspondence through an algorithm, and then combining actions of the treatment instrument with the image data precisely; and step (C) real-time checking the image data and assisting guidance and navigation to perform surgeries on intraoral tissues through movement of the treatment instrument at teeth receiving treatment.

The intraoral tissue receiving treatment is a patient's mouth, including intraoral soft and hard tissues; or the intraoral tissue receiving treatment is a patient's dental model.

Step (C) includes switching a manner of checking the image data through a control device, switching that checking the image data does not vary with movement of the treatment instrument, or checking the image data varies with movement of the treatment instrument.

Step (C) includes directly making check during a surgery of treating the intraoral tissue through real-time checking the image data. Alternatively, after step (B), the method further includes step (B-1), using surgical planning software to combine the image data to plan a surgical planning data file, so that the system provides guide and alert according to the surgical planning data file during the clinical treatment in step (C). The surgical planning data file made in step (B-1) is planned clinically by a doctor, to serve as an assisted guidance and navigation basis of the surgery on the intraoral tissue. Alternatively, the surgical planning data file made in step (B-1) is planned by a trainer (teacher) and/or a trainee (student), to serve as an implementation guide basis of the surgery of treating the intraoral tissue.

Further, after step (B-1), the method further includes step (B-2), transferring the surgical planning data file to make a surgical guide plate.

The advantages of the present invention lie in using known CT photography and an optical positioning system to track medical appliances, which uses CT photography and an optical positioning device to decide relevant direction, angle and depth for a dental surgery, also provides selection information of treatment instruments, and real-time provides CT images of intraoral tissues around the treatment instruments, to achieve the function of assisting guidance and navigation during the clinical surgery, and during the oral treatment of the doctor, the existing use habits of the physicians are not affected and accurate and convenient auxiliary information is provided, and attention is paid to using the treatment instrument in physical environments such as a patient's tooth or dental model. The method may not need surgical planning in applications, which greatly reduces the duration of the intraoral tissue surgical treatment through the assisted guidance and navigation method in the present invention; the method also may be applied to treatment requiring surgical planning and training or manufacturing of a surgical guide plate, thereby greatly increasing the efficacy of surgical planning and training assessment.

### BRIEF DESCRIPTION OF THE DRAWINGS

FIG. 1 is a schematic view of an implementation flow of the present invention;

FIG. 2 is a first schematic view of another implementation flow of the present invention; and

FIG. 3 is a second schematic view of another implementation flow of the present invention.

### DETAILED DESCRIPTION OF THE EMBODIMENTS

Detailed content and technical description about the present invention are further described with embodiments, but it should be understood that, the embodiments are only exemplary, instead of being construed as limitations to implementation of the present invention.

The present invention discloses an assisted guidance and navigation method in intraoral surgeries, which is a method using CT photography and an optical positioning system to track medical appliances.

The CT photography is a known technique, and is not the focus of the patent, which is not repeated herein. U.S. Pat. No. 6,675,040 “Optical Object Tracking System” discloses an optical detection system, used for recording positions of instruments connected with optically detectable objects in space, which, through camera systems in combination with data processors, image scan data, and computers and associated graphic display, could provide tracking of instruments, objects, patients, and apparatus in a surgical, diagnostic, or treatment setting; and the following patent technology discloses related improved technologies, and thus the technology of tracking medical appliances through an optical positioning system is a known technology, and is not the focus of the patent, which is not repeated herein.

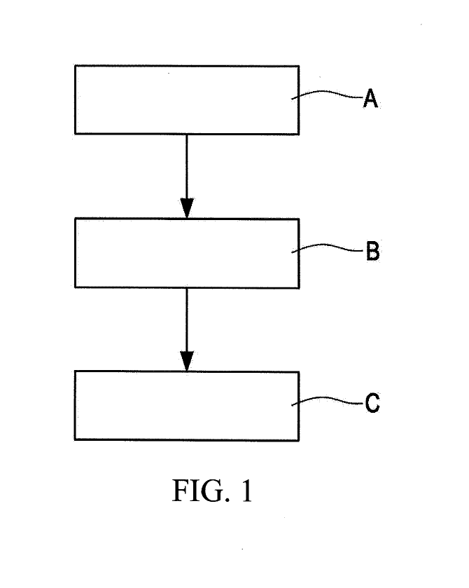

FIG. 1 is a schematic view of an implementation flow of the present invention. The assisted guidance and navigation method in intraoral surgeries according to the present invention at least includes the following steps.

Step (A): provide an optical positioning treatment instrument and an optical positioning device at an intraoral tissue receiving treatment. The intraoral tissue receiving treatment is a patient's intraoral soft and hard tissues, or a patient's dental model. The existing intraoral tissue treatment manners mostly rely on clinical experience and surgical techniques of the doctor, for some treatment cases, if the doctor assesses that treatment can be directly made, the optical positioning treatment instrument and the optical positioning device can be used in the mouth of the patient receiving treatment; and the doctor can make assessment by first making the patient's tooth model or clinical surgery, and setting the optical positioning treatment instrument and the optical positioning device on the completed teeth model, to make the following pre-surgery planning or pre-surgery simulation and comparison.

Step (B): obtain image data of the intraoral tissue receiving treatment through CT photography, obtain a positioning relationship between the treatment instrument and the optical positioning device, make the image data and physical space generate correspondence through an algorithm, and then combine actions of the treatment instrument with the image data precisely. By taking tooth implant as an example, loading CT photography of receiving treatment through imaging software can display the jawbone anatomical images in the patient's implant area, to help the doctor to decide the best implant orientation, and provide the doctor with a more complete dental 3D virtual environment.

Step (C): the doctor could real-time check the image data and perform assisted guidance and navigation for the intraoral tissue through movement of the treatment instrument at teeth receiving treatment according to the positioning relationship between the treatment instrument and the optical positioning device and the correspondence generated by the image data and the physical space by only moving the treatment instrument.

According to the steps of the method, the doctor, during treatment of the intraoral tissues, could focus on using the treatment instrument in physical environments such as a patient's mouth or tooth model. The method, in terms of implementation, does not affect the existing use habits of the physicians and accurate and provides convenient auxiliary information, which may not require surgical planning in applications and can reduce the duration of the intraoral tissue surgical treatment.

In terms of implementation, step (C) may include switching a manner of checking the image data through a control device, switching that checking the image data does not vary with movement of the treatment instrument, or checking the image data varies with movement of the treatment instrument. For example, switching of image data is controlled and checked through a foot switch or a manual switch, so that the image data may not vary with movement of the treatment instrument, which facilitates the doctor to make more detailed interpretation and evaluation for some CT images.

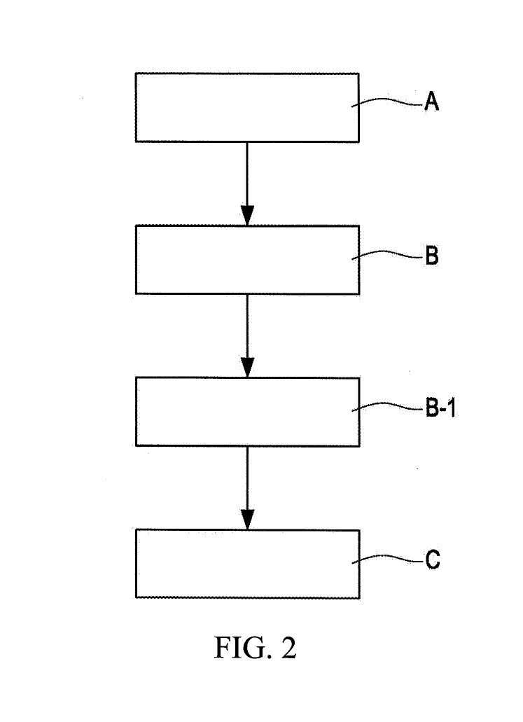

Referring to FIG. 2, after step (B), the method further includes a step (B-1), where the doctor can use surgical planning software to plan a surgical planning data file, and through pre-surgery planning, so that the system performs assisted guidance and navigation and alert according to planning data during the treatment in step (C).

The surgical planning data file made in step (B-1) is planned by a doctor, to directly serve as an assisted guidance and navigation basis of the surgery of treating the intraoral tissue. Alternatively, the surgical planning data file made in step (B-1) is planned by a trainer (teacher) and/or a trainee (student), wherein the trainee (student) could use his own planning as implementation guide practice, and the trainer (teacher) may make correction, or the trainer (teacher) provides a standard surgical planning data file to serve as an implementation assisted guidance and navigation basis of the surgery of treating the intraoral tissue, which can be applied to education and training of the intraoral tissue surgery.

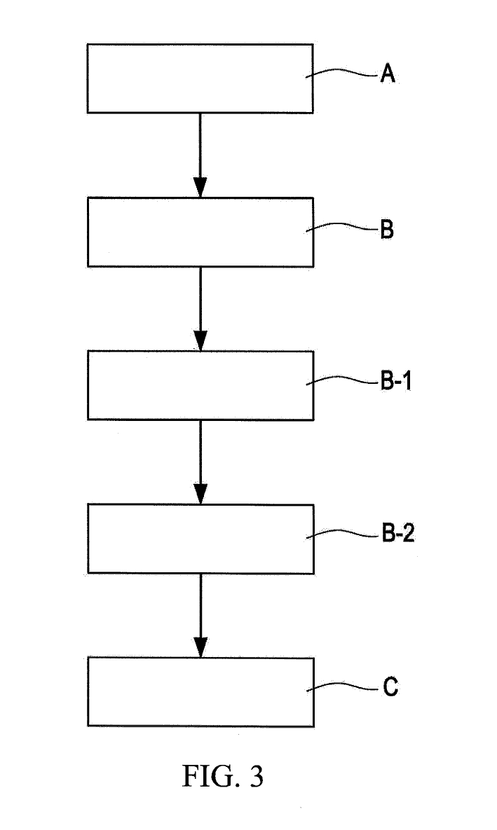

Referring to FIG. 3, after step (B-1), the method further includes a step (B-2), wherein the surgical planning data file is transferred to make a surgical guide plate. The existing technology of manufacturing a surgical guide plate may be divided into two types: rapid prototyping and numerical drilling, wherein the rapid prototyping technology is disclosed in U.S. Pat. No. 5,768,134; the numerical drilling technology is disclosed in U.S. Pat. No. 5,967,777, U.S. Pat. No 6,296,483, U.S. Pat. No. 6,814,575, and so on, the technology of manufacturing surgical guide plate is a known technology, and is not the focus of the patent, which is not repeated herein.

The present invention uses a new treatment method using CT photography and an optical positioning system to track medical appliances, which uses CT photography and an optical positioning device to decide relevant direction, angle and depth for an intraoral tissue surgery, also provides selection information of treatment instruments, and real-time provides CT images of intraoral tissues around the treatment instruments, to achieve the function of assisting guidance and navigation during the surgery, and during the oral treatment, the doctor can focus on using the treatment instrument in physical environments such as a patient's tooth or dental model, which may not need surgical planning in applications, greatly reduces the duration of the treatment, and also greatly increase the efficacy of surgical planning assessment.

The above are only preferred embodiments of the present invention, which cannot be used to define the scope of the present invention. That is, any simple equivalent variations and modifications made according to the claims and the content of the description should fall into the scope of the present invention.

What is claimed is:

1. An assisted guidance and navigation method in intraoral surgeries, which is a method using computerized tomography (CT) photography and an optical positioning system to track medical appliances, the method at least comprising:
(A) providing an optical positioning treatment instrument and an optical positioning device at an intraoral tissue receiving treatment;
(B) obtaining image data of the intraoral tissue receiving treatment through CT photography, obtaining a positioning relationship between the treatment instrument and the optical positioning device, making the image data and physical space generate correspondence through an algorithm, and then combining actions of the treatment instrument with the image data precisely; and
(C) real-time checking the image data and assisting guidance and navigation to perform a surgery on the intraoral tissue through movement of the treatment instrument at teeth receiving treatment.

2. The method according to claim 1, wherein the intraoral tissue receiving treatment is a patient's mouth.

3. The method according to claim 1, wherein the intraoral tissue receiving treatment is a patient's dental model.

4. The method according to claim 1, wherein step (C) comprises switching a manner of checking the image data through a control device, switching that checking the image data does not vary with movement of the treatment instrument, or checking the image data varies with movement of the treatment instrument.

5. The method according to claim 1, wherein step (C) comprises directly making check during a surgery on the intraoral tissue through real-time checking the image data.

6. The method according to claim 1, wherein after step (B), the method further comprises step (B-1), using surgical planning software to combine the image data to plan a surgical planning data file.

7. The method according to claim 6, wherein the surgical planning data file made in step (B-1) is planned clinically by a doctor, to serve as a guide basis of the surgery of treating the intraoral tissue in step (C).

8. The method according to claim 6, wherein the surgical planning data file made in step (B-1) is planned by a trainer and/or a trainee, to serve as an implementation assisted guidance and navigation basis of the surgery on the intraoral tissue in step (C).

9. The method according to claim 6, wherein, after step (B-1), the method further comprises step (B-2), transferring the surgical planning data file to make a surgical guide plate.
